(19) United States
(12) Patent Application Publication
(10) Pub. No.: US 2025/0366924 A1
(43) Pub. Date: Dec. 4, 2025

(54) MEDICAL AR SYSTEM FOR SURGICAL PROCEDURES AND METHOD FOR VERIFYING NAVIGATION ACCURACY

(71) Applicant: B. Braun New Ventures GmbH, Freiburg im Breisgau (DE)

(72) Inventors: Maximilian Dippel, Freiburg (DE); Jean Stawiaski, Freiburg (DE)

(21) Appl. No.: 19/226,884

(22) Filed: Jun. 3, 2025

(30) Foreign Application Priority Data
Jun. 4, 2024 (DE) 10 2024 115 529.9

(51) Int. Cl. A61B 34/10; A61B 34/00; A61B 34/20; A61B 90/00

(52) U.S. Cl. CPC A61B 34/10; A61B 34/20; A61B 34/25; A61B 90/36; A61B 2034/101; A61B 2034/2051; A61B 2034/2055; A61B 2034/2068; A61B 2034/256; A61B 2090/365

(57) ABSTRACT

A medical augmented reality system for a surgical procedure on a patient includes a visualization unit for generating a preoperative image and a real-time image with a reference point, a navigation system for capturing the visualization unit, and a control unit for generating an augmented reality overlay display from a digitized reference point from the preoperative image and the real-time image and for displaying a comparison of the augmented reality overlay display. The navigation accuracy of the medical augmented reality system can be verified with the aid of a method, a computer-readable storage medium and/or a computer program.

### CROSS-REFERENCE TO RELATED APPLICATION

This application claims priority under 35 U.S.C. § 119 to German Application No. 2024 115 529.9, filed on Jun. 4, 2024, the content of which is incorporated by reference herein in its entirety.

### FIELD

The present disclosure relates to a medical augmented reality (AR) system for a surgical, in particular neurosurgical, procedure on a patient. The AR system has a visualization unit that generates a preoperative image, in particular a 3D image, of the patient with at least one (current) reference point marked on it and a real-time image, in particular a 3D image, of the patient with the at least one (current) reference point. In addition, the AR system has a control unit that is set up to generate a digitized reference point registered for the patient from the at least one (current) reference point of the preoperative image. In addition, the present disclosure relates to a method for verifying the navigation accuracy of the medical AR system, as well as a computer-readable storage medium and a computer program.

### BACKGROUND

Traditional approaches to surgical navigation and planning rely on preoperative imaging techniques to obtain detailed representations of the surgical target areas. These methods typically involve the use of MRI (magnetic resonance imaging), CT (computed tomography), and ultrasound imaging to generate comprehensive views of the areas to be operated on. Despite the high quality of these images, one challenge is to effectively link these preoperative images to the actual situation during the surgical procedure. Well-known systems rely on external tracking systems to link the images generated preoperatively with the intraoperative reality. However, these approaches often require complicated setup processes and can restrict the surgeon's freedom of movement.

In the field of medical imaging and surgery, the integration of augmented reality (AR) technologies has enabled significant advances, particularly in the precision and efficiency of surgical procedures. Augmented reality offers a contactless way to interact with and perceive patient data. The introduction of AR into the operating room has opened up new possibilities that enable more direct and intuitive interaction with preoperative data. By overlaying digital images directly onto the surgeon's field of view, AR systems can offer seamless integration of imaging information into the surgical workflow. Despite these considerable advances, limitations remain in terms of the accuracy of the overlay and the ability to account for changes in real time. The accuracy of positional determination and the stability of image overlay are critical factors that influence the usefulness of AR in surgery. In high-precision applications such as neurosurgery, minor inaccuracies in the surgical procedure can have significant effects with sometimes catastrophic consequences for patient safety. In addition, the effective use of AR systems in surgery requires seamless integration into the surgical workflow, which poses a challenge as the systems often place an additional cognitive burden on the surgeon and interaction with the systems during the procedure must be intuitive and uninterrupted.

Despite considerable advances in medical imaging and AR technology, there is still a need for improved methods and systems that enable reliable and user-friendly integration of AR into surgical procedures without adversely affecting patient safety. In particular, there is a need for systems that offer greater navigational accuracy and better adaptability to intraoperative changes without interrupting the surgical workflow or increasing the cognitive load on the surgeon.

### SUMMARY

Therefore, the present disclosure is based on the task of avoiding or at least mitigating the disadvantages described above and, in particular, providing a medical AR system and a method that enables precise and reliable integration of AR into surgical procedures.

This task is solved by a medical AR system, a method for verifying navigation accuracy, or a computer-readable storage medium and/or a computer program. Advantageous embodiments are explained below.

The disclosure therefore initially relates to a medical AR system for surgical, in particular neurosurgical, procedures on patients. The medical AR system features a visualization unit.

The visualization unit is designed to generate and provide a preoperative (3D) image of the patient with at least one (current/momentary) reference point marked on it (i.e., on the patient). The visualization unit is designed to generate and provide a real-time (3D) image of the patient, for example in the form of a video feed, with at least one (current) reference point/marker. The medical AR system features a navigation system. The navigation system is designed to capture the position and orientation of the visualization unit in a global coordinate system relative to the patient. The medical AR system features a visual display device for displaying visual information for the surgical procedure. The medical AR system has a control unit. The control unit is set up and prepared to generate and store at least one digitized reference point registered to the patient from the at least one (current) reference point of the preoperative (3D) image of the patient. The control unit is additionally set up and prepared to generate an AR overlay display with the at least one digitized reference point and the real-time image of the patient (which has the at least one current reference point), to display this on the visual display device, and to compare the at least one digitized reference point with the at least one (current) reference point of the real-time image. This is particularly useful for verifying the navigation accuracy of the medical AR system during the procedure.

In other words, the system has a visualization unit that generates real-time images of the patient with at least one, preferably at least two or three, marked current reference points and a preoperative momentary 3D image of the patient and the at least one, preferably at least two or three, current reference points. The visualization unit of the medical AR system is designed to generate the preoperative 3D image of the patient, including at least one current reference point in this preoperative 3D image, which is then transmitted to a navigation system and serves as the basis for the navigation processes in the subsequent surgical procedure. A navigation system captures the position and orientation of the visualization unit in relation to the patient in a global coordinate system. The visual display device displays visual information for the surgical procedure, while a control unit generates and stores digital reference points from the current reference points of the preoperative image.

According to a central aspect of the disclosure, the control unit generates an AR overlay display that simultaneously displays (overlays) the digitized reference points with the current reference points of the real-time image of the patient. This enables the user or, preferably, the system to continuously verify and (automatically) adjust the navigation accuracy during the procedure. A visual display device is used to display this visual information and presents the augmented images and data in a form that is understandable and usable for the user. The ability to seamlessly integrate real-time data and images into the surgeon's field of view improves decision-making and precision during the procedure. The control unit is designed to generate and store digitized reference points from the patient's preoperative 3D images. These digitized points serve as anchors for overlaying the AR displays and are crucial for maintaining spatial consistency between the virtual information and the physical patient. The ability to generate and store these reference points accurately is fundamental to the accuracy and reliability of the entire system.

Real-time verification of navigation accuracy, especially contactless verification, improves surgical precision, which in turn increases the safety and effectiveness of the procedure and addresses the challenge of maintaining accuracy in dynamic surgical environments. These high-resolution 3D images are essential for planning and performing the procedure, as they provide a three-dimensional basis for navigation and orientation. The reference points from preoperative images are matched with the current images taken during the procedure to ensure continuous accuracy and timeliness of visual information. This allows planning and navigation information to be communicated during all phases of a surgical procedure, enabling the user to verify the accuracy of medical system registration and navigation, particularly during the procedure, with the aid of augmented reality and minimal manual interaction.

In a further preferred embodiment of the disclosure, the control unit may be set up and prepared to determine a distance between the at least one digitized reference point and the at least one current reference point of the real-time image as a navigation error of the medical AR system and preferably to correct it automatically.

In other words, the control unit can, especially before the actual surgical procedure begins, determine and preferably quantify a deviation between the digitized reference points and the current reference points on the patient in the current (video feed) image and output/display this on the display device.

This enables more precise and dynamic adjustment and verification of navigation accuracy during the surgical procedure. The control unit, which is already configured to generate an AR overlay display with the digitized reference point and the real-time image of the patient showing the current reference point, is thus able to quantify the spatial discrepancy between the digitized and the current reference point. This quantification of the distance enables the system to provide an objective measurement of the deviation in real time, allowing the user to make corrections/adjustments, especially before the surgical procedure begins. The identification and quantification of the navigation error as a specific distance measurement between the reference points provides a direct feedback loop that enables the surgical team to monitor and adjust the precision of the procedure.

The implementation of these specific communication mechanisms between the components, in particular between the control unit and the visual display device, enables seamless integration of real-time monitoring of navigation accuracy into the workflow of the surgical procedure. This innovation leads to increased safety and efficiency in surgical procedures by minimizing the risk of navigation errors and enabling more precise alignment with surgical targets. The ability to identify and correct navigation errors, preferably in real time, represents a significant advance in medical imaging and navigation that has the potential to significantly improve patient outcomes and reduce the challenges faced by surgeons during complex procedures.

In a further advantageous aspect of the disclosure, the navigation system can be designed to (additionally) capture a position and orientation of a medical instrument tip in a global coordinate system relative to the patient, and the control unit is set up and designed to generate the at least one digitized reference point using the position and orientation of the medical instrument tip.

This extension enables direct and precise tracking of the instrument tip in real time, which is formed, for example, with a separate marker/tracker. The control unit may be configured to generate a digitized reference point using the position and orientation of the medical instrument tip. This makes it possible to update and refine the position of the instrument tip in relation to the preoperative planning data and the current intraoperative conditions, and to select reference points on the patient to be digitized using the instrument tip.

Alternatively or additionally, the control unit can be set up and prepared to select and digitize the reference points using a laser pointer and/or a focus point of the visualization unit. The use of a laser pointer or focus point enables significantly more precise localization and selection of reference points on the patient. The focus point, which is part of the visualization unit, allows the reference points to be positioned with high accuracy by pointing directly at the relevant location on the patient's body. Accordingly, the control unit can generate a digitized reference point, which is then fed into the augmented reality (AR) overlay display. This display, which is displayed on the visual display device, combines digital information with the real world.

In a further or alternative advantageous embodiment of the disclosure, the control unit may be set up and designed to automatically generate the at least one digitized reference point using the at least one current reference point of the preoperative 3D image of the patient generated by the visualization unit. This means that the control unit can be set up to automatically generate digitized reference points based on the preoperative 3D image of the patient generated by the visualization unit and the current reference points/markers positioned on the patient in this image, i.e., without additional manual selection of the current reference points on the patient.

This has the advantage that the control unit can automatically generate at least one digitized reference point based on the current reference point identified in the preoperative 3D image.

In a further preferred embodiment of the disclosure, the medical AR system may comprise an AR headset and/or a 2D monitor and/or a 3D monitor and/or a virtual reality (VR) headset as the display device. In other words, the visual AR overlay display from the real-time image and the digitized reference points can be displayed alternatively or additionally via a VR or AR headset worn by the user on their head.

The integration of an AR headset or VR headset as a display device allows the user to enjoy an immersive, augmented reality experience in which digital information is displayed directly in the user's field of vision. This promotes intuitive interaction with the visual data and supports more precise navigation and orientation during the surgical procedure by reducing the need to look away from the surgical site. The use of a 3D monitor as a display device offers an alternative visualization method that allows the surgical team to view the three-dimensional anatomical structures and the positioning of reference points in real time, facilitating collective assessment and decision-making during the procedure.

In a further preferred embodiment of the disclosure, the navigation system may comprise an infrared-based tracking system or an electromagnetic tracking system or an optical machine vision tracking system.

In other words, the visualization unit is captured in particular via the navigation system, for example using an infrared-based, electromagnetic, or optical tracking system. In all cases, the tracking system captures the relative position of the visualization system in relation to the patient. A tracking system can also be used, which calculates the relative position of the visualization system to the patient from kinematic data/information from the robot or robotic arm with which the visualization unit is connected.

In other words, the navigation system can be either an infrared-based tracking system, an electromagnetic tracking system, or an optical machine vision tracking system. In an infrared-based tracking system, communication takes place via infrared signals that are reflected by markers attached to the patient, enabling highly accurate positioning and orientation of the visualization unit relative to the patient. An electromagnetic tracking system uses electromagnetic fields to capture the spatial position and orientation of the visualization unit. An optical machine vision tracking system, on the other hand, uses image processing algorithms to detect and track the position of the markers on the patient.

According to another advantageous aspect of the disclosure, the visualization unit may be configured as a surgical microscope, a surgical exoscope, a surgical endoscope, or an optical camera.

The AR system according to the present disclosure can preferably enable a 3D perception of the displayed AR information. Furthermore, the entire AR system can preferably run in a simulated environment or in a digital twin.

The present disclosure further relates to a method for verifying a navigation accuracy of a medical AR system, comprising the steps of setting at least one, preferably at least two, current reference points/markers on a patient, generating and providing a preoperative 3D image of the patient with the at least one current reference point by a visualization unit, generating and storing at least one digitized reference point from the at least one current reference point of the preoperative 3D image of the patient by a control unit, generating and providing a real-time image of the patient with the at least one current reference point by the visualization unit, generating an AR overlay display with the at least one digitized reference point and the real-time image of the patient, which has the at least one current reference point, outputting the AR overlay display by a visual display device, and comparing the at least one digitized reference point with the at least one current reference point of the real-time image in order to verify the navigational accuracy of the medical AR system.

According to a further advantageous embodiment of the disclosure, the method may comprise an additional step of calculating a navigation error of the medical AR system from a distance/deviation between the at least one digitized reference point and the at least one current reference point of the real-time image.

By calculating the distance between the respective reference points, the control unit can determine a navigation error. The ability to immediately detect and correct deviations minimizes the risk of errors during the procedure, improves surgical outcomes, and increases patient safety. This feature also enables objective evaluation of the performance of the medical AR system by providing a quantifiable measurement of navigation accuracy.

According to a further embodiment, the navigation system can capture a position and orientation of a medical instrument tip in a global coordinate system relative to the patient, and the step of generating and storing the at least one digitized reference point can be performed using the position and orientation of the medical instrument tip.

The present disclosure further relates to a computer-readable storage medium comprising instructions that, when executed by a computer, cause the computer to perform the steps of the method disclosed herein.

Furthermore, the present disclosure continues to relate to a computer program comprising instructions which, when executed by a computer, cause the computer to perform the steps of the method disclosed herein.

### BRIEF DESCRIPTION OF THE DRAWING FIGURES

The disclosure is explained in more detail below with reference to preferred embodiments with the aid of the accompanying figures. The following is shown:

FIG. 1 shows an exemplary perspective side view of a medical AR system according to a preferred exemplary embodiment of the present disclosure;

FIG. 2 shows an example of a real-time image of a patient with a plurality of reference points marked thereon and an example of a medical instrument according to the preferred exemplary embodiment of the present disclosure;

FIG. 3 shows an example of a preoperative 3D image of the patient and the plurality of marked reference points being captured by a visualization unit, which are automatically transmitted to the navigation system as digitized reference points, in accordance with the preferred exemplary embodiment of the present disclosure;

FIG. 4 shows an example of a perspective AR overlay display with a plurality of digitized reference points and a real-time image of a draped patient, and

FIG. 5 is a flowchart of a method for verifying navigation accuracy of the medical AR system according to a preferred embodiment of the present disclosure.

The figures are schematic in nature and serve only to aid understanding of the revelation. Identical elements are marked with the same reference signs. The features of the various embodiments can be used interchangeably and in any combination.

### DETAILED DESCRIPTION

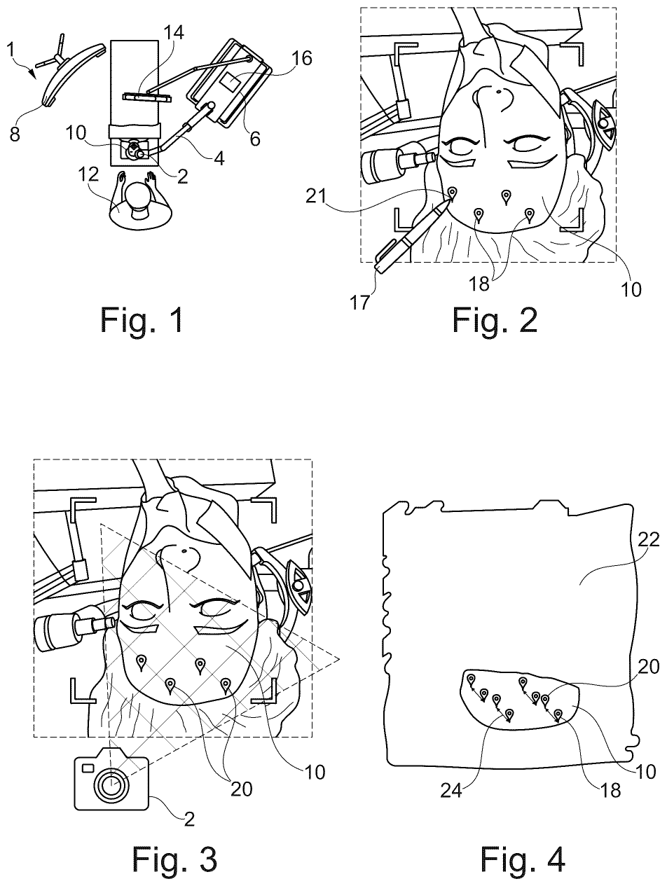

The present disclosure is described below with reference to an advantageous embodiment with reference to FIGS. 1 through 5.

FIG. 1 is an exemplary perspective side view of a medical AR system 1 according to a preferred exemplary embodiment of the present disclosure. The AR system 1 features a movable visualization unit 2, in particular in the form of a surgical microscope, which is connected to a movable robotic arm 4 of a medical robot 6 in order to adjust both the position (x, y, z) and the orientation of the visualization unit 2 in the room relative to the patient by controlling the robotic arm 4. The user/operator 12 monitors the position of the visualization unit 2, which generates a real-time image of a patient 10 and of reference points/markings placed on the patient prior to surgery. Furthermore, the AR system 1 has a navigation system 8 which, by means of an integrated navigation camera, in this case a stereo camera, captures the position and orientation of the visualization unit 2 relative to a (registered) patient 10. The AR system 1 also features a display device 14, for example in the form of a 2D monitor, and a control unit 16. The control unit 16 is adapted such that it generates an AR overlay display with at least one digitized reference point 20 and the real-time image of the patient 10, which has the at least one current reference point 18, and displays it on the display device 14.

FIG. 2 is an example of a real-time image of patient 10 with a plurality of (current) reference points 18 marked on it and an example of a medical instrument 17, which has an instrument tip 21 that can be tracked by the navigation system 8, in accordance with the preferred exemplary embodiment of the present disclosure. The real-time image of patient 10, which is captured by the visualization unit 2 and can be displayed as a real-time video transmission on the display device 14, thus represents a real-time image of patient 10 including the real-time/realistic markings/reference points 18 and, preferably, also a real-time image of a medical instrument 17.

FIG. 3 shows an example of a preoperative 3D image of patient 10 and the plurality of marked reference points 18 being captured by a visualization unit 2, which are automatically transmitted to a navigation system 8 as digitized reference points 20, according to the preferred exemplary embodiment of the present disclosure. The digitized reference points 20 are generated by the control unit 16 of the medical AR system 1 based on the captured real-time reference points 18.

FIG. 4 is an example of a perspective AR overlay display with a plurality of digitized reference points 20 and the real-time image of a patient 10 with a plurality of real-time reference points 18. In this illustration, patient 10 is draped with a medical sheet/cover 22 for subsequent surgical intervention, and only a portion (in this illustration, the forehead) of patient 10, which has the reference points 18, is not covered by the medical sheet 22. The characteristic anatomical landmarks of patient 10 are therefore no longer easily accessible to the visualization unit 2. Nevertheless, augmented reality can be used to display the previously digitized reference points 20 to the user 12, who is then able to visually compare them with the reference points 18 marked on the patient 10 and displayed in real time in the display device 14. The shift between the digitized reference points 20 and the respective real-time reference points 18 is then the navigation error 24 of the AR system 1, which is preferably indicated or highlighted separately on the display device 14.

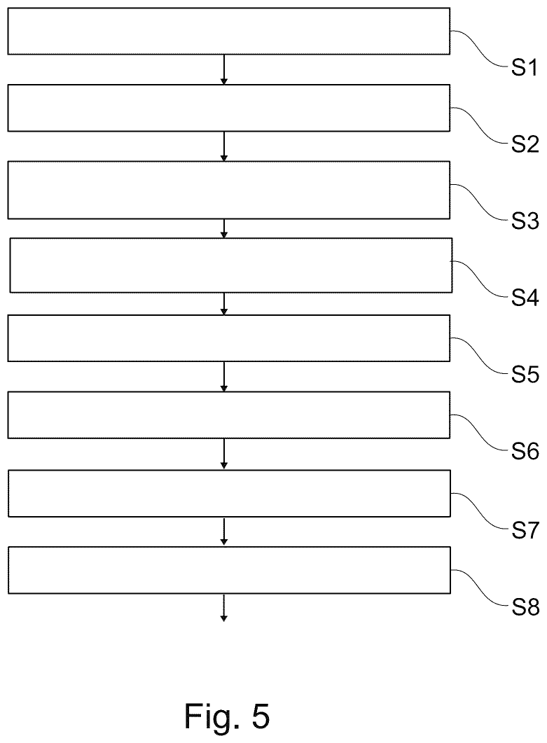

FIG. 5 is a flowchart of a (computer-implemented) method for verifying a navigation accuracy of the medical AR system 1 according to a preferred embodiment of the present disclosure.

In a first step S1, at least one current reference point 18 is set on a patient 10 prior to surgery. This is done using a medical marker, for example. In a second step S2, a preoperative 3D image of patient 10 with at least one current reference point 18 is generated by a visualization unit 2 and made available. In the third step, at least one digitized reference point 20 is generated from the at least one current reference point 18 of the preoperative 3D image of the patient 10 by the control unit 16 and stored. At the same time, before or alternatively after step S3, in the fourth step S4, a real-time image of patient 10 with at least one current reference point 18 is generated and provided by the visualization unit 2. Subsequently, in the fifth step, an AR overlay display is generated with the at least one digitized reference point 20 and the real-time image of the patient 10, which has the at least one current reference point 18, and in the sixth step S6, it is output on the visual display device 14. In the last step S7, the control unit 16 compares the at least one digitized reference point 20 with the at least one current reference point 18 of the real-time image.

In a further step S8, a navigation error 24 of the medical AR system 1 can be calculated by the control unit 16 from a distance between the at least one digitized reference point 20 and the at least one current reference point 18 of the real-time image.

In a further preferred embodiment of the disclosure, the navigation system 8 can capture a position and orientation of a medical instrument tip 21 in a global coordinate system relative to the patient 10, and step S3 of generating and storing the at least one digitized reference point 20 can be performed using the position and orientation of the medical instrument tip 21.

### LIST OF REFERENCE SIGNS

1 Medical AR system
2 Visualization unit
4 Movable robotic arm
6 Medical robot
8 Navigation system
10 Patient
12 Users
14 Display device
16 Control unit
17 Medical instrument
18 Current reference point
20 digitized reference point
21 Instrument tip
22 Medical sheet
24 Navigation error
S1 Step Setting the at least one current reference point
S2 Step Generating and providing a preoperative 3D image
S3 Step Generating and saving at least one digitized reference point
S4 Step Generating and providing a current image of the patient
S5 Step Generating an AR overlay display
S6 Step Outputting the AR overlay display by the display device
S7 Step Comparing the at least one digitized reference point with the at least one current reference point
S8 Step Calculating a navigation error of the medical AR system

1. A medical augmented reality system for a surgical procedure on a patient, the medical augmented reality system comprising:
a visualization unit configured to generate and provide a preoperative image of the patient with at least one reference point marked on the preoperative image, and to generate and provide a current image of the patient with the at least one reference point;
a navigation system configured to capture a position and orientation of the visualization unit in a global coordinate system relative to the patient,
a visual display device configured to display visual information for the surgical procedure; and
a control unit configured to generate and store at least one digitized reference point registered on the patient from the at least one reference point of the preoperative image of the patient,
the control unit being additionally configured to generate an augmented reality overlay display with the at least one digitized reference point and the current image of the patient, displaying the augmented reality overlay display on the visual display device, and comparing the at least one digitized reference point with the at least one reference point of the current image.

2. The medical augmented reality system according to claim 1, wherein the control unit is configured to determine a distance between the at least one digitized reference point and the at least one reference point of the current image as a navigation error of the medical augmented reality system.

3. The medical augmented reality system according to claim 2, wherein the control unit is configured to correct the navigation error automatically.

4. The medical augmented reality system according to claim 1, wherein:
the navigation system is configured to capture a position and orientation of a medical instrument tip in a global coordinate system relative to the patient, and
the control unit is configured to generate the at least one digitized reference point using the position and orientation of the medical instrument tip.

5. The medical augmented reality system according to claim 1, wherein the control unit is configured to generate the at least one digitized reference point with a laser pointer and/or a focal point of the visualization unit.

6. The medical augmented reality system according to claim 1, wherein the control unit is configured to automatically generate the at least one digitized reference point based on the at least one reference point of the preoperative image of the patient generated by the visualization unit.

7. The medical augmented reality system according to claim 1, wherein the visual display device comprises at least one of:
an augmented reality headset;
a 3D monitor; or
a virtual reality headset.

8. The medical augmented reality system according to claim 1, wherein the navigation system comprises an infrared-based tracking system.

9. The medical augmented reality system according to claim 1, wherein the navigation system comprises an electromagnetic tracking system.

10. The medical augmented reality system according to claim 1, wherein the navigation system comprises an optical machine vision tracking system.

11. A method for verifying navigation accuracy of a medical augmented reality system, the method comprising the steps of:
setting at least one reference point on a patient;
generating and providing a preoperative image of the patient with the at least one reference point by a visualization unit;
generating and storing at least one digitized reference point from the at least one reference point of the preoperative image of the patient by a control unit;
generating and providing a real-time image of the patient with the at least one reference point by the visualization unit;
generating an augmented reality overlay display with the at least one digitized reference point and the real-time image of the patient;
outputting the augmented reality overlay display via a visual display device; and
comparing the at least one digitized reference point with the at least one reference point of the real-time image by the control unit.

12. The method according to claim 11, further comprising the step of calculating a navigation error of the medical augmented reality system from a distance between the at least one digitized reference point and the at least one reference point of the real-time image by the control unit.

13. The method according to claim 12, further comprising the step of capturing a position and orientation of a medical instrument tip in a global coordinate system relative to the patient, wherein the step of generating and storing the at least one digitized reference point is performed using the position and orientation of the medical instrument tip.

14. A computer-readable storage medium and/or a computer program comprising instructions which, when executed by a computer, cause the computer to perform the method according to claim 11.
